(19) United States
(12) Patent Application Publication
(10) Pub. No.: US 2003/0059834 A1
(43) Pub. Date: Mar. 27, 2003

(54) Methods to generate and identify monoclonal antibodies to a large number of human antigens

(76) Inventor: Nancy T. Chang, Houston, TX (US)

Correspondence Address: TANOX, INC., 10301 STELLA LINK, HOUSTON, TX, 77025, US

(21) Appl. No.: 10/061,910

(22) Filed: Feb. 1, 2002

Related U.S. Application Data
(60) Provisional application No. 60/265,701, filed on Feb. 1, 2001.

(51) Int. Cl. G01N 33/53; C12P 21/04; C07K 16/00

(52) U.S. Cl. 435/7.1; 435/69.1; 435/70.21; 435/320.1; 530/388.1

(57) ABSTRACT

A method of determining the antigens encoded by a genomic or cDNA library is disclosed. Dendritic or other antigen presenting cells are transfected with DNA fragments in a vector which includes a signal peptide coding sequence and an sequence which encodes a peptide binding to a receptor on the antigen presenting cell. The expressed DNA fragments are secreted under control of the signal peptide, and bind to a cell surface receptor. The antigen presenting cells are used to generate monoclonal antibodies. The monoclonal antibodies may be screened by cloning the same fragments into a display vector containing a transmembrane domain thereby displaying the expressed proteins on the surface of a host cell. The monoclonal antibodies are screened against these displayed proteins for a positive match.

### BACKGROUND

We describe an efficient method for generating pools of antibodies for high throughput screening, which can be used for therapeutics and/or diagnostics. This method may be used to screen for antigens that induce antibody responses.

### BACKGROUND

We describe an efficient method for generating pools of antibodies for high through-put screening, which can be used for therapeutics and/or diagnostics. This method may be used to screen for antigens that induce antibody responses.

In order to keep pace with the volume of sequence data, the field of functional genomics has attempted to use different types of high throughput analysis to determine gene function. Recently, a number of techniques have been developed that are designed to link gene expression levels, or tissue or cell specific gene expression, to gene function. These include cDNA microarray and “gene chip” technology, as well as the differential display of messenger RNA (mRNA). Serial Analysis of Gene Expression (SAGE) or differential display of mRNA can identify genes that are expressed in tumor tissue but are absent in the respective normal or healthy tissue. In this way, genes which regulate expression or transcription, or are otherwise needed for tumor growth, can be separated from ubiquitously expressed genes that are less likely chance to be useful for small drug screening or gene therapy projects. Gene chip technology has the potential to quickly allow the monitoring of expression of a large number of genes through the measurement of mRNA expression levels in cells. mRNA expression patterns of cells cultured under a variety of conditions can be analyzed and compared. DNA microarray chips with 40,000 non-redundant human genes have been produced (Editorial (1998) Nat. Genet. 18(3):195-7.). However, measuring mRNA expression levels with these techniques is primarily designed for screening cancer cells for tumor genes, and not for screening for specific gene functions or for screening for antigenicity.

The challenge in functional genomics is to develop and refine all the above-described techniques and integrate their results with existing data in a well-developed database that provides for the development of a picture of gene function and a means for this knowledge to be put to use in the development of novel medicinal products. The current technologies have limitations and do not necessarily result in functional data. Therefore, there is a need for a method that allows for direct measurement of the function of a single gene from a collection of genes (gene pools or individual clones) in a high throughput setting in appropriate in vitro assay systems and animal models.

It is fairly well established that many pathological conditions, such as infections, cancer, autoimmune disorders, etc., are characterized by the inappropriate expression of certain molecules. These molecules thus serve as “markers” for a particular pathological or abnormal condition. Apart from their use as diagnostic “targets”, i.e., materials to be identified to diagnose these abnormal conditions, the molecules serve as reagents that can be used to generate diagnostic and/or therapeutic agents.

Preparation of such materials, of course, presupposes a source of the reagents used to generate these. Purification from cells is one laborious, far from sure method of doing so. Another method is the isolation of nucleic acid molecules which encode a particular marker, followed by the use of the isolated encoding molecule to express the desired molecule. To date, two strategies have been employed for the detection of such antigens, in e.g., human tumors. One approach is exemplified by, e.g., dePlaen et al., Proc. Natl. Sci. USA 85: 2275 (1988), where several hundred pools of plasmids of a cDNA library obtained from a tumor are transfected into recipient cells, such as COS cells, or into antigen-negative variants of tumor cell lines. Transfectants are screened for the expression of tumor antigens via their ability to provoke reactions by antitumor cytolytic T cell clones.

The second approach, exemplified by, e.g., Mandelboim, et al., Nature 369: 69 (1994), is based on isolation of peptides which have bound to MHC-class I molecules of tumor cells, followed by reversed-phase high performance liquid chromography (HPLC). Antigenic peptides are identified after they bind to empty MHC-class I molecules of mutant cell lines, defective in antigen processing, and induce specific reactions with cytotoxic T-lymphocytes. These reactions include induction of CTL proliferation, TNF release, and lysis of target cells, measurable in an MTT assay, or a ⁵¹Cr release assay.

These two approaches to the molecular definition of antigens have the following disadvantages: first, they are enormously cumbersome, time-consuming and expensive; second, they depend on the establishment of cytotoxic T cell lines (CTLs) with predefined specificity; and third, their relevance in vivo for the course of the pathology of disease in question has not been proven. So far only a very few new antigens have been identified in human tumors. See, e.g., van der Bruggen et al., Science 254: 1643-1647 (1991); Brichard et al., J. Exp. Med. 178: 489-495 (1993); Coulie, et al., J. Exp. Med. 180: 35-42 (1994); Kawakami, et al., Proc. Natl. Acad. Sci. USA 91: 3515-3519 (1994).

At present, there are no efficient methods for high through-put screening for antigenic proteins from a given cDNA source. The present invention provides such a method.

### SUMMARY OF THE INVENTION

The invention relates to a method of screening large numbers of antigens simultaneously by their ability to generate antibodies. Once the antibodies are generated, expression cloning and functional assays may be used to characterize the individual antibodies and the nature of each antigen that generated them. For example, a mast cell specific cDNA library can be used to generate antibodies to all of the expressed proteins in a single immunization.

The method involves the cloning of a library of cDNAs or genomic DNA isolated from a chosen source, e.g., mast cells, lymphocytes, etc., into a fusion vector, such as the one depicted in FIG. 1, and a display vector, such as the one depicted in FIG. 2. The fusion vector comprises a signal peptide that directs the secretion of the expressed protein and a region that allows binding to a dendritic cell. This binding region can be, e.g., an Fc region, thus allowing binding and internalization for antigen processing. Alternatively, the binding region can comprise a ligand for a dendritic cell receptor. The display vector, such as pSecTM-FV, comprises a signal sequence, an epitope tag, and a transmembrane domain.

The fusion vector containing the cDNA library is transduced into monocyte derived immature dendritic cells. The binding region of the secreted protein binds to its receptor on the dendritic cell, followed by antigen processing and expression of the protein on the cell surface. The pool of dendritic cells, each containing a fusion vector, is then injected into an animal, such as a mouse. The protein expressed on the surface of the dendritic cell, if antigenic, will elicit B-cell activation and antibody formation. The B cells from spleens or lymph nodes of the mice are then subjected to fusion with myeloma cells according to standard hybridoma techniques. Alternatively, primary B cells could be functionally identified and their individual immunoglobulin genes cloned. The monoclonal antibodies are then expanded for characterization. The supernatants of the hybridomas are pooled and screened as described below.

Simultaneously, the same cDNA library is also cloned into a display vector. The cDNA sequences are displayed on the surface of the transfected host cells by virtue of the transmembrane domain fused to the 3′ end of the expressed protein. Host cells can be any mammalian cell that provides reasonable expression of the vector construct, e.g., 293 cells, CHO cells, etc. The transfected cells which express the protein on the cell surface are then sorted by using, e.g., a commercially available antibody that binds to the tag sequence. The sorted cells are then seeded into microwell plates for screening.

Pooled supernatants from the hybridomas generated from the dendritic cell immunization are screened against the display vector library allowing rapid identification of multiple antigens that generated antibodies in the immunized animal.

### BRIEF DESCRIPTION OF THE FIGURES

FIG. 1 schematically depicts the Fc fusion vector, pSec-Fc

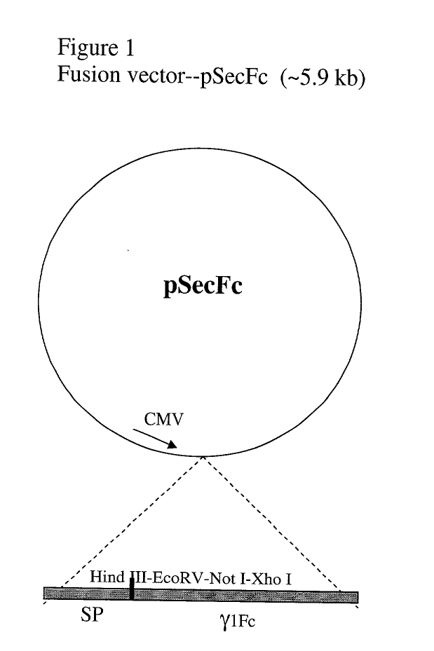

FIG. 2 schematically depicts the Display vector, pSecTM-FV

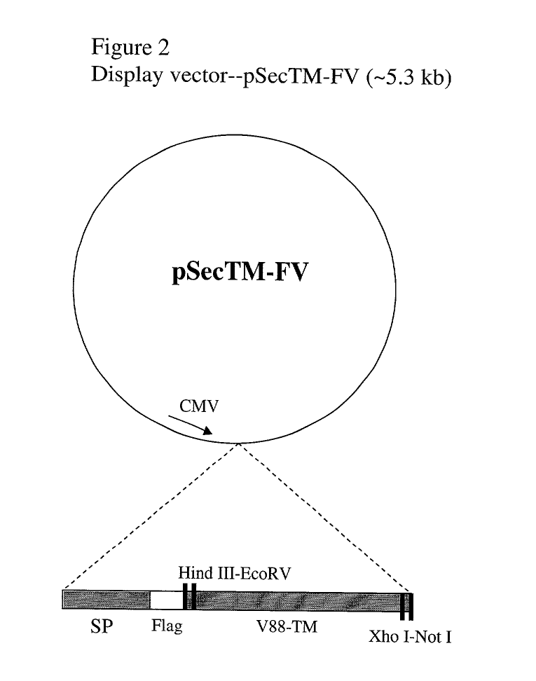

FIG. 3 depicts the nucleic acid sequence of the insertion region of the Display Vector pSecTM-FV

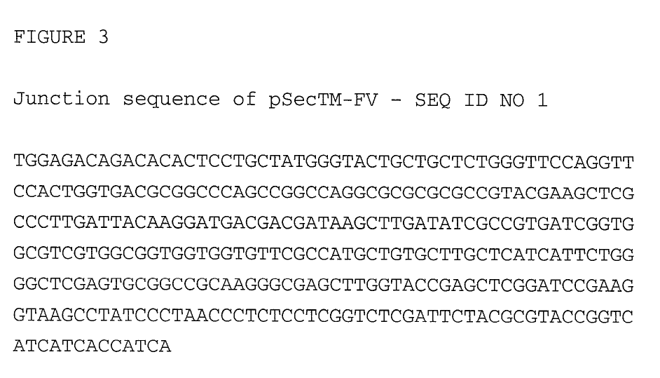

### DETAILED DECRIPTION

Constructs

There are a variety of commercially available vectors that can be used as a starting framework for engineering the fusion vector and display vector. These include, e.g., the TOPO vector system (Invitrogen, Gaithersburg, Md.), which utilizes the CMV promoter; pMSG, from Pharmacia (Piscataway, N.J.), which uses the glucocorticoid-inducible promoter of the mouse mammary tumor virus long terminal repeat to drive expression of the cloned gene; pSVL (Pharmacia, Piscataway), which utilizes the SV40 late promoter; pEF-1a; and pUB which utilizes the ubiquitin promoter.

cDNAs or genomic DNAs from different sources may be cloned into an expression vector with a signal peptide at 5′ end and a targeting moiety at the 3′ end to facilitate the secretion of the protein and the binding to the antigen presenting cells. The targeting moiety could be an Fc of an IgG molecule that binds to the Fc receptor on antigen presenting cells or other ligands that can bind to their receptors on the antigen presenting cells. The cDNAs library is transfected or transduced into purified or enriched antigen presenting cells in vitro.

Fusion constructs containing a signal peptide, DNA fragments, and a targeting sequence are used to transduce or transfect antigen-presenting cells. These cells are then used to immunize mice for hybridoma production. Positive clones can be identified using cells transfected with cDNAs fused to sequences encoding transmembrane anchoring sequence and screened by fluorescence activated cell sorting (FACS) or immunofluorescene staining or by differentially screening using normal vs. diseased tissues/cells or displaying peptide or proteins. Antigens inducing the antibody response can then be characterized by normal methods of functional analysis.

Vectors for use in constructing either the fusion vector or the display vector include expression vectors, adenoviral vectors, and retroviral vectors. Mammalian expression vectors are described in EP-A-0367566, and in U.S. Pat. No. 5,350,683, incorporated by reference herein. The vectors may also be derived from retroviruses.

Adenoviral serotypes 2 and 5 have been extensively used for vector construction. Bett et al., Proc. Nat. Acad. Sci. U.S.A., 1994, 91: 8802-8806 have used an adenoviral type 5 vector system with deletions of the E1 and E3 adenoviral genes. The 293 human embryonic kidney cell line has been engineered to express E1 proteins and can thus transcomplement the E1-deficient viral genome. The virus can be isolated from 293 cell media and purified by limited dilution plaque assays (Graham, F. L. and Prevek, L. In Methods in Molecular Biology: Gene Transfer and Expression Protocols, Humana Press 1991, pp. 109-128).

AAV-based vectors may be used to transduce cells with nucleic acids of interest. See West et al. (1987) Virology 160:38-47; Carter et al. (1989) U.S. Pat. No. 4,797,368; Carter et al. WO 93/24641 (1993); Kotin (1994) Human Gene Therapy 5:793-801; and Muzyczka (1994) J. Clin. Invst. 94:1351. Samulski (1993) Current Opinion in Genetic and Development 3:74-80. Recombinant AAV vectors deliver foreign nucleic acids to a wide range of mammalian cells (Hermonat & Muzycka (1984) Proc Natl Acad Sci USA 81:6466-6470; Tratschin et al. (1985) Mol Cell Biol 5:3251-3260), integrate into the host chromosome (Mclaughlin et al. (1988) J Virol 62: 1963-1973), and show stable expression of the transgene in cell and animal models (Flotte et al. (1993) Proc Natl Acad Sci USA 90:10613-10617). Moreover, unlike retroviral vectors, AAV vectors are able to infect non-dividing cells (Podsakoff et al. (1994) J Virol 68:5656-66; Flotte et al. (1994) Am. J. Respir. Cell Mol. Biol. 11:517-521). Proteins produced in mammalian cells often do not have the solubility and secretion problems encountered in bacterial expression.

The signal sequence may be a polynucleotide encoding an amino acid sequence that initiates transport of a protein across the membrane of the endoplasmic reticulum. Signal sequences which will be useful in the invention include antibody light chain signal sequences, e.g., antibody 14.18 (Gillies et. al., 1989, Jour. of Immunol. Meth., 125:191-202); antibody heavy chain signal sequences, e.g., the MOPC141 antibody heavy chain signal sequence (Sakano et al., 1980, Nature 286:5774); the signal sequence of IL-7 described in U.S. Pat. No. 4,965,195; the signal sequence for IL-2 receptor described in Cosman et al., Nature 312:768 (1984); the IL-4 signal peptide described in EP 367,566; the type I IL-1 receptor signal peptide described in U.S. Pat. No. 4,968,607; and the type II IL-1 receptor signal peptide described in EP 460,846; or any other signal sequences which are known in the art (see for example, Watson, 1984, Nucleic Acids Research 12:5145).

Signal sequences have been well characterized in the art and are known typically to contain 16 to 30 amino acid residues, and may contain greater or fewer amino acid residues. A typical signal peptide consists of three regions: a basic N-terminal region, a central hydrophobic region, and a more polar C-terminal region. The central hydrophobic region contains 4 to 12 hydrophobic residues that anchor the signal peptide across the membrane lipid bilayer during transport of the nascent polypeptide. A detailed discussion of signal peptide sequences is provided by yon Heijne (1986) Nucleic Acids Res., 14:4683 (incorporated herein by reference). As would be apparent to one of skill in the art, the suitability of a particular signal sequence for use in the fusion vector may require some routine experimentation. Additionally, one skilled in the art is capable of creating a synthetic signal peptide following the rules presented by yon Heijne, referenced above, and testing for the efficacy of such a synthetic signal sequence by routine experimentation. A signal sequence is also referred to as a “signal peptide” these terms having meanings synonymous to signal sequence may be used herein.

The Fc region of an immunoglobulin is the amino acid sequence for the carboxyl-terminal portion of an immunoglobulin heavy chain constant region. As known, the heavy chains of the immunoglobulin subclasses comprise four or five domains: IgM and IgE have five heavy chain domains, and IgA, IgD and IgG have four heavy chain domains. The Fc region of IgA, IgD and IgG is a dimer of the hinge-CH2—CH3 domains, and in IgM and IgE it is a dimer of the hinge-CH2—CH3—CH4 domains. Further the CH3 domain of IgM and IgE is structurally equivalent to the CH2 domain of IgG, and the CH4 domain of IgM and IgE is the homolog of the CH3 domain of IgG (see, W. E. Paul, ed., 1993, Fundamental Immunology, Raven Press, New York, N.Y., which publication is incorporated herein by reference). Any of the known Fc regions would be useful as the Fc region of the fusion vector. However, it is important that the binding sites for certain proteins be deleted from the Fc region during the construction of the fusion vector, e.g., the cysteine residues present in the Fc regions which are responsible for binding to the light chain of the immunoglobulin should be deleted or substituted with another amino acid, such that these cysteine residues do not interfere with the proper folding of the Fc region. In the same manner, transmembrane domain sequences, such as those present in IgM, should be deleted such that these sequences do not result in misdirecting the protein expressed from the fusion vector to the membrane as a transmembrane protein.

The currently preferred class of immunoglobulin from which the Fc region is derived is immunoglobulin gamma-1, because it has been well characterized and is efficiently secreted from most cell types. The Fc region of the other subclasses of immunoglobulin gamma (gamma-2, gamma-3 and gamma-4) would function equally well in the fusion vector.

As is apparent from the above discussion of Fc regions, the Fc regions from the other classes of immunoglobulins, IgA, IgD, IgE, and IgM, would also be useful as the Fc region of the fusion vector. Further, deletion constructs of these Fc regions, in which one or more of the constant domains are deleted would also be useful. One of ordinary skill in the art could prepare such deletion constructs using well known molecular biology techniques.

Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the binding region and the heterologous protein sequence, so that the binding region may be cleaved away from the heterologous moiety following purification. Methods for fusion protein expression and purification are discussed in Ausubel, F. M. et al. (1995 and periodic supplements) Current Protocols in Molecular Biology, John Wiley & Sons, New York, N.Y., ch 10. A variety of commercially available kits may also be used to facilitate expression and purification of fusion proteins.

Generation of Monocyte-Derived Immature Antigen Presenting Cells

Monocytes were purified from PBMC by immunomagnetic depletion (monocyte-enrichment cocktail containing MAbs against CD2, CD3, CD16, CD19, CD56, CD66b and glycophorin A; StemSep™ from StemCell Technologies, Vancouver, Canada). Monocyte (>90% CD14⁺) preparations devoid of neutrophilic granulocytes, platelets, lymphocytes and NK cells were subsequently cultured in serum-free culture medium, StemSpan™ (StemCell Technologies), supplemented with 10 ng/ml GM-CSF and 20 ng/ml IL-4 (both cytokines from PeproTech, Rocky Hill, N.J., USA) at 37° C./5%CO₂ during 6-7 days. These monocytes were seeded at a cell density of 1×10⁶/2 ml/10 cm² polystyrene surface (coated with 12 mg/mil 10 cm² poly-hydroxyethyl-methacrylate; Sigma) and fresh GM-CSF/IL-4 was added at day 2 and 5. After 6-7 days, the nonadherent cells (with a dendritic morphology) were collected and displayed the following (flow cytometry, see below) phenotypic profile: CD1 a⁺, CD14⁻, CD40⁺, C80⁺, CD83⁻, CD86⁺, HLA-DR⁺ and mannose receptor⁺⁺.

Immunization:

Transduced antigen-presenting cells are injected into rodents (e.g. mouse, rats, etc.) to induce an antibody response and hybridomas are prepared. Alternatively, B cells could be isolated from the spleen or lymph nodes of the immunized mice, cultured in vitro and then antibodies tested for specificity to antigens. The B cells are then isolated for single cell polymerase chain reaction (PCR) to clone the immunoglobulin genes. The antigen presenting cells transduced with cDNA libraries express and secrete the proteins. These proteins then bind to the receptor through the receptor-binding moiety at the c-terminus. The proteins are internalized by a receptor-mediated uptake and then processed intracellularly before being presentedon the antigen presenting cells. This process will enhance antigen presentation for induction of immune responses to the protein. After a period of one to three months, the mice are sacrificed and spleens are excised for preparation of splenocytes. The cells are then fused with mouse myeloma cells to generate hybridomas secreting antibodies.

Alternatively, mice could be immunized with the fusion vector cDNA library and GM-CSF, which expands the antigen presenting cells, subcutaneously by direct injection, e.g., with the Genegun (BioRad, Hercules, Calif.).

For each hybridoma, single cell suspensions are prepared from the spleen of an immunized mouse and fused with Sp2/0 myeloma cells. 5×10⁸ of the Sp2/0 and 5×10⁸ spleen cells are fused in a medium containing 50% polyethylene glycol (M. W. 1450) (Kodak, Rochester, N.Y.) and 5% dimethylsulfoxide (Sigma Chemical Co., St. Louis, Mo.). The cells are then adjusted to a concentration of 1.5×10⁵ spleen cells per 200 μl of the suspension in Iscove medium (Gibco, Grand Island, N.Y.), supplemented with 10% fetal bovine serum, 100 units/ml of penicillin, 100 μg/ml of streptomycin, 0.1 mM hypoxanthine, 0.4 μM aminopterin, and 16 μM thymidine. Two hundred μl of the cell suspension is added to each well of about fifty 96-well microwell plates. After about ten days, culture supernatants are withdrawn for screening for reactivity with the display vector library.

Screening of Antibodies:

Supernatants from hybridoma culture or serum from mice will be tested by incubating with the transfected cells and then analyzed by FACS or immunofluorescence staining to identify positive clones. Mock-transfected cells may be used as negative controls. Alternatively, cDNAs can be cloned into phage vectors and display the antigens on their surface for screening antibodies. To minimize the generation of antibodies to most abundant proteins, cDNA subtraction and/or normalization can be performed before generating cDNA libraries. To screen for antibodies to tumor antigens or antigens in disease states, differential screening can be used on normal tissues (cells) vs. diseased or normal tissues (cells) vs. tumor tissues (cells).

Wells of Immulon 2 (Dynatech Laboratories, Chantilly, Va.) microwell plates are coated by adding 50 μl of each display vector clone. After the coating solution was removed by flicking the plate, 200 μl of BLOTTO (non-fat dry milk) in PBS is added to each well for one hour to block the non-specific sites. An hour later, the wells are washed with a buffer PBST (PBS containing 0.05% Tween 20). Fifty microliters of culture supernatants from each fusion well are collected and mixed with 50 μl of BLOTTO and then added to the individual wells of the microwell plates. After one hour of incubation, the wells are washed with PBST. The bound murine antibodies are then detected by reaction with horseradish peroxidase (HRP)conjugated goat anti-mouse IgG (Fc specific) (Jackson ImmunoResearch Laboratories, West Grove, Pa.) and diluted at 1:2,000 in BLOTTO. Peroxidase substrate solution containing 0.1% 3,3,5,5 tetramethyl benzidine (Sigma) and 0.0003% hydrogen peroxide (Sigma) is added to the wells for color development for 30 minutes. The reaction is terminated by addition of 50 μl of 2M H₂SO₄ per well. The OD at 450 nm of the reaction mixture is read with a BioTek ELISA Reader (BioTek Instruments, Winooski, Vt.).

The culture supernatants from the positive wells are further characterized after positive wells are cloned by limiting dilution. The selected hybridomas are grown in spinner flasks and the spent culture supernatant collected for antibody purification by protein A affinity chromatography.

We claim:

1. A method of generating monoclonal antibodies to a large number of mammalian antigens comprising:
a. generating a plurality of gene fragments from a genomic or a cDNA library;
b. cloning the plurality of gene fragments into a fusion vector comprising a promoter sequence, a signal peptide sequence, a cloning site, and a binding region sequence wherein the binding region encoded is specific for an antigen presenting cell membrane receptor;
c. transducing or transfecting immature antigen-presenting cells with the library of fusion vector constructs containing the plurality of gene fragments;
d. introducing the transduced or transfected antigen-presenting cells into a mammalian host;
e. isolating B-cells from the mammalian host and preparing hybridomas to generate monoclonal antibodies.

2. The method of claim 1, wherein the binding region comprises an immunoglobulin Fc region.

3. The method of claim 2, wherein the Fc region is gamma-1 Fc.

4. The method of claim 1, wherein the binding region comprises a ligand for a dendritic cell receptor.

5. The method of claim 1, wherein the cDNA gene fragments are prepared by subtractive hybridization.

6. The method of claim 1, wherein the cDNA is mast cell specific.

7. A method of screening the monoclonal antibodies of claim 1, comprising:
a. cloning the same plurality of gene fragments used in step (b) of claim 1 into a display vector generating a display library, said display vector comprising a promoter sequence, a signal sequence, an epitope tag, a cloning site, and a transmembrane domain sequence;
b. transducing or transfecting host cells that express said display vector with the display library and seeding multiwell plates with individual transduced or transfected host cells;
c. screening the monoclonal antibodies against the microwell plates and detecting binding through generation of a positive signal.

8. The method of claim 7, wherein the epitope tag sequence is selected from glutathione S-transferase (GST), maltose binding protein (MBP), thioredoxin (Trx), calmodulin binding peptide (CBP), 6-His, FLAG, c-myc, and hemagglutinin (HA).

9. The method of claim 8, wherein the epitope tag is FLAG.

10. The method of claim 7, wherein the display vector is pSECTM-FV.

11. A fusion vector encoding a signal peptide, a promoter, and a binding region specific for an antigen presenting cell membrane receptor, wherein at least one polynucleotide fragment from a cDNA library has been inserted downstream from the promoter.

12. A display vector encoding a signal peptide, an epitope tag sequence, a promoter, and a transmembrane domain, wherein at least one polynucletide fragment from the cDNA library of claim 11 has been inserted downstream from the promoter.

13. An antigen presenting cell comprising a fusion vector selected from the pool of vector constructs of claim 11.

14. A transformed cell comprising a display vector selected from the pool of vector constructs of claim 12.

15. The fusion vector of claim 11, wherein the binding region comprises an Fc region.

16. The fusion vector of claim 15, wherein the Fc region is gamma-1 Fc.

17. The fusion vector of claim 11, wherein the binding region comprises a ligand for a dendritic cell receptor.

18. The fusion vector of claim 11, wherein the cDNA is prepared by subtractive hybridization.

19. The fusion vector of claim 11, wherein the cDNA is mast cell specific.

20. The display vector of claim 12, wherein the epitope tag is selected from glutathione S-transferase (GST), maltose binding protein (MBP), thioredoxin (Trx), calmodulin binding peptide (CBP), 6-His, FLAG, c-myc, and hemagglutinin (HA).

21. The display vector of claim 20, wherein the epitope tag is FLAG.

22. A method of generating monoclonal antibodies to a large number of mammalian antigens comprising:
a. generating a plurality of gene fragments from a genomic or a cDNA library;
b. cloning the plurality of gene fragments into a fusion vector comprising a promoter sequence, a signal peptide sequence, a cloning site, and a binding region sequence wherein the binding region encoded is specific for an antigen presenting cell membrane receptor;
c. introducing the fusion vector cDNA library into a mammal subcutaneously in combination with GM-CSF;
d. isolating B-cells from the mammalian host and preparing hybridomas to generate monoclonal antibodies.

23. The method of claim 22, wherein the binding region comprises an Fc region.

24. The method of claim 23, wherein the Fc region is gamma-1 Fc.

25. The method of claim 22, wherein the binding region comprises a ligand for a dendritic cell receptor.

26. The method of claim 22, wherein the cDNA is prepared by subtractive hybridization.

27. The method of claim 22, wherein the cDNA is mast cell specific.

28. The method of claim 22, wherein the mammal is a mouse.
